(19) United States
(12) Patent Application Publication
(10) Pub. No.: US 2007/0100377 A1
(43) Pub. Date: May 3, 2007

(54) Providing multiple signal modes for a medical device

(75) Inventors: Randolph K. Armstrong, Houston, TX (US); Steven M. Parnis, Pearland, TX (US); Timothy L. Scott, Sugar Land, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: CYBERONICS, INC.

(21) Appl. No.: 11/260,951

(22) Filed: Oct. 28, 2005

(51) Int. Cl. A61N 1/36

(52) U.S. Cl. 607/2

(57) ABSTRACT

A method, system, and an apparatus are provided for providing multiple stimulation modes for a medical device, such as an implantable medical device. The method includes applying a first electrical signal to a nerve of a patient during a primary time period. The method further includes applying a second electrical signal to the nerve of the patient during a secondary time period in which the first electrical signal is not applied. The secondary electrical signal may provide a reduced level of stimulation that improves a therapeutic effect and/or reduces a side effect associated with the first electrical signal.

### BACKGROUND OF THE INVENTION

1. Field of the Invention

This invention relates generally to medical devices and, more particularly, to methods, apparatus, and systems for providing a background signal using a medical device capable of treating a medical condition of a patient.

2. Description of the Related Art

The human brain resides in the cranial cavity of the skull and controls the central nervous system (CNS) in a supervisory role. The central nervous system is generally a hub of electrical and/or neural activity requiring appropriate management. For example, properly controlled electrical or neural activity enables the human brain to manage various mental and body functions to maintain homeostasis. Abnormal electrical and/or neural activity is associated with different diseases and disorders in the central and peripheral nervous systems. In addition to a drug regimen or surgical intervention, potential treatments for such diseases and disorders include implantation of a medical device in a patient for electrical stimulation of body tissue. In particular, by selectively applying therapeutic electrical signals to one or more electrodes coupled to the patient's neural tissue, an implantable medical device (IMD) may electrically stimulate a target neural tissue location. This stimulation may be used to treat a neurological disease, condition or disorder.

Therapeutic electrical signals may be used to apply an electrical signal to a neural structure of the body, and more particularly to cranial nerves such as the vagus nerve. The signal may be used to induce afferent action potentials on the nerve and thereby increase the flow of neural signals up the nerve, toward the brain. The signal may also (or alternatively) generate efferent action potentials to modulate a neural response in one or more body structures of the patient, such as any of the numerous organs innervated by efferent signals on the vagus nerve. Finally, therapeutic electrical signals may also or additionally be used to inhibit neural activity and to block neural impulses from moving up or down the nerve the nerve. As used herein, the terms “stimulate” and “modulate” are interchangeable and refer to delivery of a signal (which may comprise an electrical, magnetic, or chemical stimulus) to a target body area, regardless of whether its effects include afferent action potentials, efferent action potentials, and/or the blocking of action potentials. Therapeutic electrical stimulation of the vagus nerve has been used to treat epilepsy and depression. Vagus nerve stimulation (VNS) therapy for treatment of epilepsy is described in many U.S. Patents including U.S. Pat. Nos. 4,702,254, 4,867,164, and 5,025,807, which are incorporated herein by reference.

To provide vagus nerve stimulation to a patient, a neurostimulator device may be implanted in a target location in the patient's body. Such a neurostimulator device system may comprise an electrical signal generator, attached to an electrical lead having one or more electrodes coupled to the vagus nerve.

However, depending upon an individual patient or a particular disease being treated, efficacy of the VNS therapy may vary significantly. For instance, VNS efficacy for treatment resistant epilepsy and depression may be generalized as a first percentage of patient population having significant improvement. A second percentage of patient population may be characterized as having some improvement. The remaining percentage of patient population may experience little or no improvement. There is a need to improve the efficacy of VNS therapy for certain treatments. Further concerns include reducing any side effects during stimulation.

Neurostimulation has demonstrated the potential to treat a wide variety of neurological disorders; however, there remains a need to increase the breadth of disorders treatable by neurostimulation.

### SUMMARY OF THE INVENTION

In one aspect, the present invention comprises a method for providing multiple stimulation modes for a medical device. The method includes applying a first signal to a nerve of a patient during a primary time period. The method further includes applying a second signal to the nerve of the patient during a secondary time period in which the first signal is not applied. In one embodiment, the first signal is an electrical signal, and the second signal is an electrical signal that is different from the first signal. The nerve may comprise a cranial nerve such as a vagus nerve of the patient.

In another aspect, a neurostimulator is provided for treating a patient with a medical condition. The neurostimulator comprises an electrical signal generator to generate a first and a second electrical signal for delivery to a selected nerve of a patient. The neurostimulator may further comprise a controller operatively coupled to the electrical signal generator. The controller may be adapted to apply the first electrical signal to the selected nerve of the patient during a primary time period, and to apply the second electrical signal to the selected nerve of the patient during a secondary time period in which the first electrical signal is off.

In a further aspect, a method of providing multiple stimulation modes for a medical device comprises applying a therapeutic stimulus signal to a nerve of a patient during a first time period. The method further comprises entering a non-therapeutic mode during a second time period subsequent to the first time period and applying a background stimulus signal during at least a portion of the second time period during the non-therapeutic mode.

In another aspect of the present invention, a method of providing multiple stimulation modes for a medical device comprises applying a first stimulus signal to a nerve of a patient during a first time period. The method further comprises applying a background stimulus signal during at least a portion of the second time period during the non-therapeutic mode.

In another aspect of the present invention, a method of providing multiple stimulation modes for a medical device comprises alternatively modulating a nerve of a patient within a stimulation period using a first electrical signal during a primary treatment period and a second electrical signal during a secondary treatment period in which the first electrical signal is not applied. The first and second signals may comprise a signal that generates an afferent action potential, an efferent action potential, or a signal that blocks native action potentials (i.e., action potentials that are not induced by an exogenously applied signal).

### BRIEF DESCRIPTION OF THE DRAWINGS

The invention may be understood by reference to the following description taken in conjunction with the accompanying drawings, in which like reference numerals identify like elements, and in which:

FIGS. 1A-1D are stylized diagrams of an implantable medical device implanted into a patient's body for providing stimulation to a portion of the patient's body, in accordance with one illustrative embodiment of the present invention;

FIG. 2 is a block diagram of an implantable medical device and an external user interface that communicates with the implantable medical device, for example, to program the implantable medical device, in accordance with one illustrative embodiment of the present invention;

FIG. 3 is a block diagram of the signal generator of FIG. 2, in accordance with one illustrative embodiment of the present invention;

FIG. 4 schematically illustrates a stylized representation of an electrical signal including a first electrical signal and a second electrical signal that may be applied to a nerve, such as a vagus nerve, by the implantable medical device of FIG. 2 during a treatment ON and OFF times of a therapy, respectively, in accordance with one illustrative embodiment of the present invention;

FIG. 5 is a flowchart depiction of the background stimulation process, in accordance with one illustrative embodiment of the present invention;

FIG. 6 is a flowchart of another embodiment of providing overlaid stimulation from the implantable medical device of FIG. 2, in accordance with one illustrative embodiment of the present invention; and

FIGS. 7A-7C illustrate stylized diagrams of various randomized electrical stimulus output current signals applied by the implantable medical device of FIGS. 1 and 2 for providing stimulation, in accordance with one illustrative embodiment of the present invention.

While the invention is susceptible of various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Illustrative embodiments of the invention are described herein. In the interest of clarity, not all features of an actual implementation are described in this specification. In the development of any such actual embodiment, numerous implementation-specific decisions must be made to achieve the design-specific goals, which will vary from one implementation to another. It will be appreciated that such a development effort, while possibly complex and time-consuming, would nevertheless be a routine undertaking for persons of ordinary skill in the art having the benefit of this disclosure.

Neurostimulation is conventionally delivered as a pulsed electrical signal in discrete stimulation periods known as pulse bursts, which constitute a series of controlled electrical pulses defined by a plurality of parameters. The signal may be generated by an electrical pulse generator and applied to the nerve via a lead/electrode assembly. The parameters defining the signal may include a current magnitude, a pulse width, a pulse frequency, an on-time and an off-time, with optional ramp-up and ramp-down periods immediately before and after the on-time in which the signal is gradually increased (ramp-up) or decreased (ramp-down) in current magnitude before or after the defined magnitude during the on-time. In prior art embodiments, the parameters may be programmed as constant, non-random values.

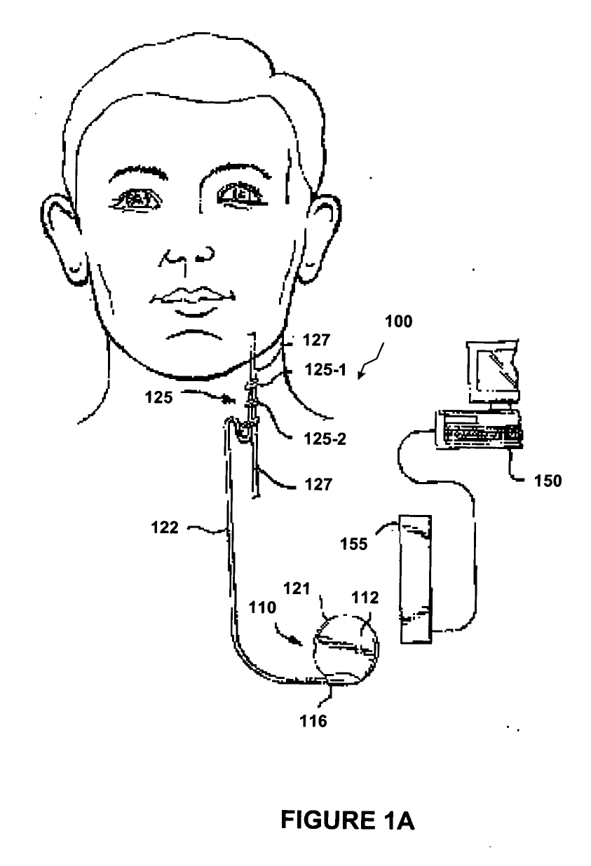

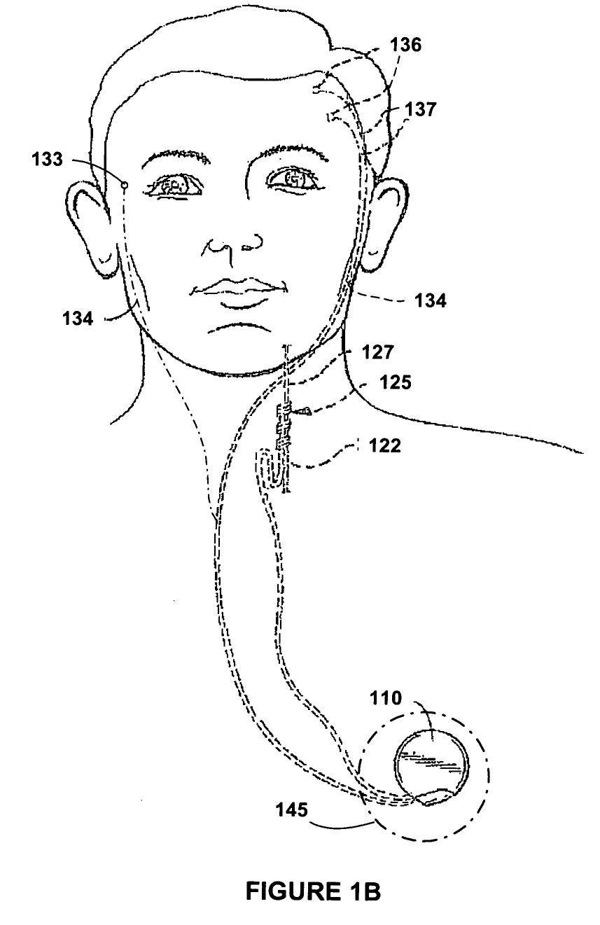

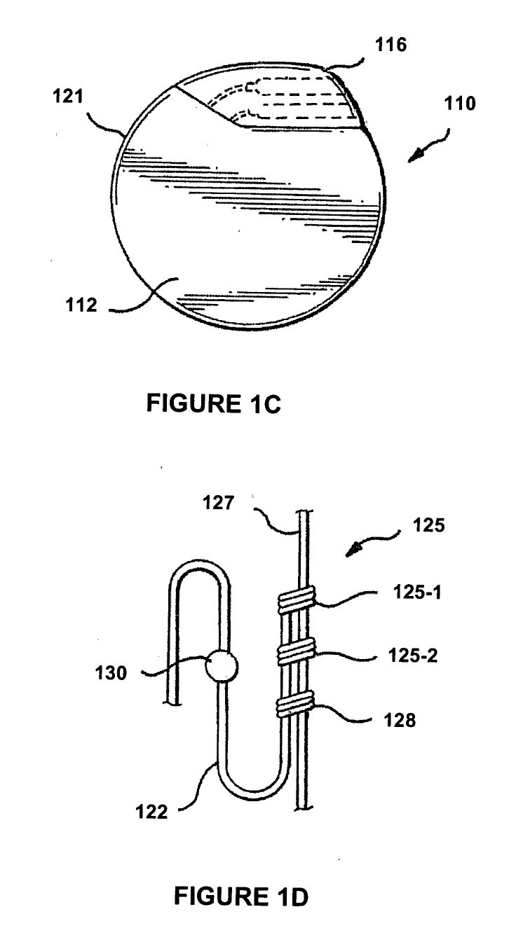

As a non-limiting example, the electrical signal may have a programmed, non-random and constant current, e.g., milliamp, a programmed frequency, e.g., 30 Hz, a programmed pulse width, e.g., 500 microseconds, a programmed current polarity, e.g., current flow from electrode 125-1 to electrode 125-2 (FIG. 1A), for a period of time, e.g., 30 seconds. The period of time in which a stimulation signal is delivered (30 seconds in the example) is referred to herein as on-time. Pulse bursts are typically separated from adjacent bursts by another period of time, e.g. 5 minutes. The period of time between delivery of stimulation signals (5 minutes in the example) is referred to herein as off-time. Ramp-up and ramp-down periods may be employed over predefined periods (typically the first few seconds or pulses of a pulse burst) to avoid discomfort sometimes associated with having the initial pulses of a burst at full amplitude. The ramping signal usually increases or decreases in a predefined, non-random manner, and the on-time portion of the pulse burst is both constant and non-random. The frequency, which is determined by a plurality of similar adjacent pulse-to-pulse intervals, is also generally a constant value, although it is known to employ a swept or randomly set value. A pulse-to-pulse interval is referred to herein as a pulse period, and is distinct from frequency in that a pulse period is independent of adjacent pulse periods, whereas a frequency, by definition, requires a plurality of similar adjacent pulse periods.

The combined signal time of a first electrical signal, including the on-time and (if present) the ramp-up and ramp-down times is referred to hereinafter as the primary time period. In embodiments where no ramp-up or ramp-down is provided, the primary period is the same as the on-time. A primary time period is typically followed by an off-time period in which no signal is applied, and the nerve is allowed to recover from the applied first electrical signal. After the off-time period elapses, the first electrical signal is again applied to the nerve for another primary time period, followed by another off-time period with no signal. This process may be repeated until altered by a healthcare provider programming the system. The on-time and the primary time period together comprise the duty cycle of the neurostimulation system.

Some embodiments of the present invention provide for applying a first electrical signal from a medical device to a nerve of a patient during a first time period in which the first electrical signal modulates the electrical activity (i.e., afferent and efferent action potentials) on the nerve, followed by a second electrical signal applied to the nerve during a second time period in which the nerve is allowed to rest and/or recover from the first electrical signal. The second electrical signal may be a sub-threshold signal that is insufficient to generate exogenous afferent or efferent action potentials on the nerve or to block native signals on the nerve, or it may comprise a modulating signal capable of generating afferent and/or efferent action potentials, or of blocking native signals. Where the second electrical signal is a sub-threshold signal the second time period is a non-stimulation time period in which the electrical activity on the nerve comprises solely native electrical activity. Regardless of whether a sub-threshold signal or a modulating signal is applied during the second time period, however, the second electrical signal is intended to reinforce and/or supplement a desired therapeutic effect of the first electrical signal, either by facilitating recovery of the nerve fibers from the first electrical signal, generating additional (exogenously induced) electrical activity on the nerve, or both.

The medical device may be an implantable medical device that is capable of providing an electrical signal to modulate the electrical activity on the nerve during the second time period to maintain a therapeutic effect of the first signal applied during a first time period. Some embodiments of the present invention provide for methods, apparatus, and systems to provide a first electrical signal to a nerve of a patient during a primary time period and a second electrical signal during a secondary time period in which the first electrical signal is not applied to the nerve of the patient. In certain embodiments the nerve comprises a cranial nerve, and more preferably a vagus nerve. The primary time period may refer to a time period in which a pulse burst (with optional ramp-up and ramp-down periods) is applied to the nerve. The secondary time period may refer to a time period in which the nerve is conventionally allowed to recover from the stimulation of the pulse burst applied during the primary time period. By modulating the electrical activity of the nerve during the secondary time period, the second electrical signal may maintain or enhance a therapeutic effect of the first electrical signal during the secondary time period. In this way, the second electrical signal provides background stimulation to a nerve, such as the vagus nerve (cranial nerve X) from an IMD, such as a neurostimulator for treating a disorder or medical condition.

Embodiments of the present invention may be employed to provide a second electrical signal at a low level, e.g., at a level that is substantially imperceptible to a patient, during a secondary period that may include a portion of the off-time of the first signal. A second electrical signal provided during an off-time of the first signal may be referred to hereinafter as “background” stimulation or modulation. For example, an IMD may apply a second electrical signal having a reduced frequency, current, or pulse width relative to the first electrical signal during off-time of the first period, in addition to the first electrical signal applied during a primary period. Without being bound by theory, applying a background electrical signal may allow the first electrical signal to be reduced to level sufficient to reduce one or more side effects without reducing therapeutic efficacy.

In some embodiments of the present invention, the first and second time periods at least partially overlap, and a second electrical stimulation signal may be applied during at least a portion of the first time period. In a more particular embodiment, the second time period only partially overlaps the first, and the second electrical stimulation signal is applied during a portion of the first time period, and continues during a period in which the first signal is not applied. This type of stimulation is referred to hereinafter as “overlaid” stimulation or modulation. Overlaid and/or background stimulation embodiments of the invention may increase efficacy of a stimulation therapy, reduce side effects, and/or increase tolerability of the first signal to higher levels of stimulation. An exemplary IMD that may be implanted into a patient's body for providing a signal to a portion of the patient's body is described below according to one illustrative embodiment of the present invention. FIGS. 1A-1D depict a stylized implantable medical system 100 for implementing one or more embodiments of the present invention. FIGS. 1A-1D illustrate an electrical signal generator 110 having a main body 112 comprising a case or shell 121 (FIG. 1A) with a header 116 (FIG. 1C) for connecting to leads 122. The electrical signal generator 110 is implanted in the patient's chest in a pocket or cavity formed by the implanting surgeon just below the skin (indicated by a dotted line 145, FIG. 1B), similar to the implantation procedure for a pacemaker pulse generator.

A stimulating nerve electrode assembly 125, preferably comprising an electrode pair, is conductively coupled to the distal end of an insulated, electrically conductive lead assembly 122, which preferably comprises a pair of lead wires (one wire for each electrode of an electrode pair). Lead assembly 122 is conductively coupled at its proximal end to the connectors on the header 116 (FIG. 1C) on case 121. The electrode assembly 125 may be surgically coupled to a vagus nerve 127 in the patient's neck or at another location, e.g., near the patient's diaphragm. Other cranial nerves may also be used to deliver the electrical neurostimulation signal. The electrode assembly 125 preferably comprises a bipolar stimulating electrode pair 125-1, 125-2 (FIG. 1D), such as the electrode pair described in U.S. Pat. No. 4,573,481 issued Mar. 4, 1986 to Bullara. Suitable electrode assemblies are available from Cyberonics, Inc., Houston, Tex. as the Model 302 electrode assembly. However, persons of skill in the art will appreciate that many electrode designs could be used in the present invention. The two electrodes are preferably wrapped about the vagus nerve, and the electrode assembly 125 may be secured to the nerve 127 by a spiral anchoring tether 128 (FIG. 1D) such as that disclosed in U.S. Pat. No. 4,979,511 issued Dec. 25, 1990 to Reese S. Terry, Jr. and assigned to the same assignee as the instant application. Lead assembly 122 is secured, while retaining the ability to flex with movement of the chest and neck, by a suture connection 130 to nearby tissue.

In one embodiment, the open helical design of the electrode assembly 125 (described in detail in the above-cited Bullara patent), which is self-sizing and flexible, minimizes mechanical trauma to the nerve and allows body fluid interchange with the nerve. The electrode assembly 125 preferably conforms to the shape of the nerve, providing a low stimulation threshold by allowing a large stimulation contact area with the nerve. Structurally, the electrode assembly 125 comprises two electrode ribbons (not shown), of a conductive material such as platinum, iridium, platinum-iridium alloys, and/or oxides of the foregoing. The electrode ribbons are individually bonded to an inside surface of an elastomeric body portion of the two spiral electrodes 125-1 and 125-2 (FIG. 1D), which may comprise two spiral loops of a three-loop helical assembly. The lead assembly 122 may comprise two distinct lead wires or a coaxial cable whose two conductive elements are respectively coupled to one of the conductive electrode ribbons. One suitable method of coupling the lead wires or cable to the electrodes 125-1 and 125-2 comprises a spacer assembly such as that disclosed in U.S. Pat. No. 5,531,778, although other known coupling techniques may be used.

The elastomeric body portion of each loop is preferably composed of silicone rubber, and the third loop 128 (which typically has no electrode) acts as the anchoring tether 128 for the electrode assembly 125.

In certain embodiments of the invention, sensors such as eye movement sensing electrodes 133 (FIG. 1B) may be implanted at or near an outer periphery of each eye socket in a suitable location to sense muscle movement or actual eye movement. The electrodes 133 may be electrically connected to leads 134 implanted via a catheter or other suitable means (not shown) and extending along the jaw line through the neck 10 and chest tissue to the header 116 of the electrical signal generator 110. When included in systems of the present invention, the sensing electrodes 133 may be utilized for detecting rapid eye movement (REM) in a pattern indicative of a disorder to be treated, as described in greater detail below. The detected indication of the disorder can be used to trigger active stimulation.

Other sensor arrangements may alternatively or additionally be employed to trigger active stimulation. Referring again to FIG. 1B, EEG sensing electrodes 136 may optionally be implanted and placed in spaced-apart relation on the skull, and connected to leads 137 implanted and extending along the scalp and temple, and then connected to the electrical signal generator 110 along the same path and in the same manner as described above for the eye movement electrode leads 134. In alternative embodiments, temperature-sensing elements and/or heart rate sensor elements may be employed to trigger active stimulation.

In contrast to active stimulation embodiments, other embodiments of the present invention utilize passive stimulation to deliver a continuous, periodic or intermittent electrical signal to the vagus nerve according to a programmed on/off duty cycle without the use of sensors to trigger therapy delivery. Both passive and active stimulation may be combined or delivered by a single IMD according to the present invention. Either or both modes may be appropriate to treat the particular disorder diagnosed in the case of a specific patient under observation.

The electrical signal generator 110 may be programmed with an external computer 150 using programming software of the type copyrighted by the assignee of the instant application with the Register of Copyrights, Library of Congress, or other suitable software based on the description herein, and a programming wand 155 to facilitate radio frequency (RF) communication between the computer 150 (FIG. 1A) and the pulse generator 110. The wand 155 and software permit non-invasive communication with the generator 110 after the latter is implanted. The wand 155 is preferably powered by internal batteries, and provided with a “power on” light to indicate sufficient power for communication. Another indicator light may be provided to show that data transmission is occurring between the wand and the generator.

By providing the stimulation therapy, the electrical signal generator 110 may treat a disorder or a medical condition. A generally suitable form of neurostimulator for use in the method and apparatus of the present invention is disclosed, for example, in U.S. Pat. No. 5,154,172, assigned to the same assignee as the present application. A commercially available example of such a neurostimulator is the NeuroCybernetic Prosthesis (NCP®, Cyberonics, Inc., Houston, Tex., the assignee of the present application). Certain parameters of the electrical signal generated by the electrical signal generator 110 are programmable, such as be means of an external programmer in a manner conventional for implantable electrical medical devices.

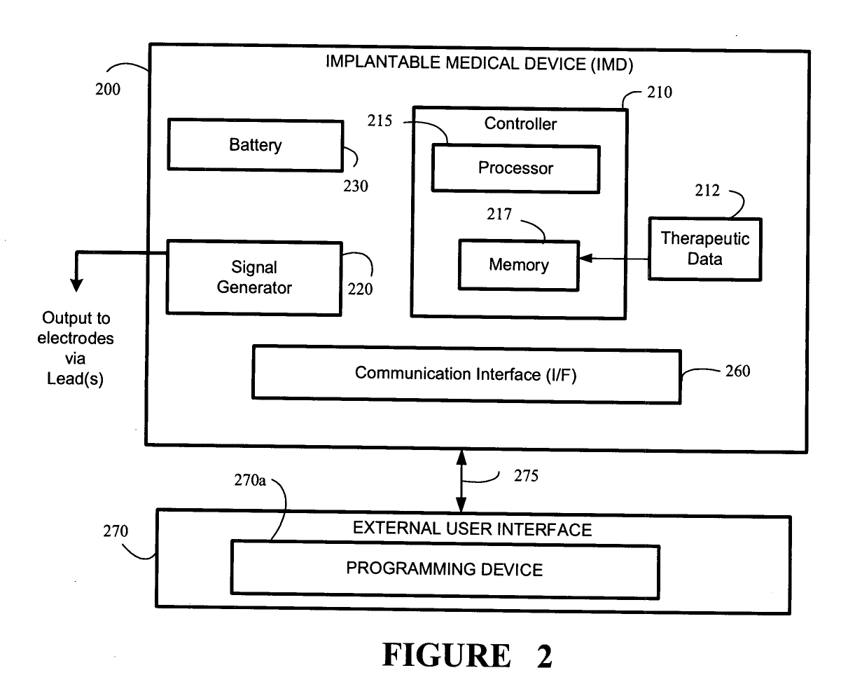

Turning now to FIG. 2, a block diagram is provided depicting an IMD 200 and an external user interface (I/F) 270, in accordance with one illustrative embodiment of the present invention. The IMD 200 may be used to provide electrical stimulation to body tissue, such as nerve tissue, to treat various disorders, such as epilepsy, depression, bulimia, etc. The IMD 200 may be used to treat neuromuscular, neuropsychiatric, cognitive, autonomic, sensory disorders, and other medical conditions.

The IMD 200 may be coupled to various leads, such as lead assembly 122, shown in FIG. 1. Electrical signals from the IMD 200 may be transmitted via the leads 122 to stimulation electrodes associated with the electrode assembly 125. In addition, where sensors are employed, signals from sensor electrodes may travel by leads, such as leads 122, 134 and/or 137, to the IMD 200.

The IMD 200 may comprise a controller 210 that is capable of controlling various aspects of the operation of the IMD 200. The controller 210 is capable of receiving therapeutic data 212 including internal data and/or external data to deliver the therapeutic electrical signal to at least one target portion of the human body. For example, the controller 210 may receive manual instructions from an operator externally, or it may perform stimulation based on internal calculations and protocols programmed into or resident in the IMD 200. The controller 210 is preferably capable of affecting substantially all functions of the IMD 200.

The controller 210 may comprise various components, such as a processor 215, a memory 217, and other structures conventional known to those skilled in the art having benefit of the present disclosure. The processor 215 may comprise one or more microcontrollers, microprocessors, etc., that are capable of performing various executions of software components. The memory 217 may comprise various memory portions where the therapeutic data 212 and a number of types of data (e.g., internal data, external data instructions, software codes, status data, diagnostic data, etc.) may be stored and retrieved. The memory 217 may comprise random access memory (RAM), dynamic random access memory (DRAM), electrically erasable programmable read-only memory (EEPROM), flash memory, etc. In one embodiment, the memory 217 may comprise RAM and Flash memory components.

The IMD 200 may also comprise an electrical signal generator 220. The signal generator 220 is capable of generating and delivering a variety of electrical neurostimulation signals to one or more electrodes via leads. A number of lead assemblies 122 may be coupled to the IMD 200. Therapy may be delivered to the lead(s) by the electrical signal generator 220 based upon instructions from the controller 210. The electrical signal generator 220 may comprise various circuitry, such as stimulation signal generators, and other circuitry that receives instructions relating to the type of stimulation to be performed. The electrical signal generator 220 is capable of delivering a controlled current neurostimulation signal over the leads. In one embodiment, the controlled current neurostimulation signal may refer to a prescribed or pre-determined current to a neural tissue of a patient.

The IMD 200 may also comprise a battery 230. The battery 230 may comprise one or more cells, voltage regulators, etc., to provide power for the operation of the IMD 200, including delivering stimulation. The battery 230 may comprise a power supply source that in some embodiments is rechargeable. The battery 230 provides power for the operation of the IMD 200, including electronic operations and the stimulation function. The battery 230, in one embodiment, may comprise a lithium/thionyl chloride cell or, more preferably, a lithium/carbon monofluoride (LiCFx) cell. It will be apparent to persons of skill in the art that other types of power supplies, e.g., high charge-density capacitors, may also be used instead of (or in addition to) the battery 230.

The IMD 200 also comprises a communication interface (I/F) 260 capable of facilitating communications between the IMD 200 and various devices. The communication interface 260 is capable of providing transmission and reception of electronic signals to and from the external user interface 270. The external user interface 270 may be a handheld device, preferably a handheld computer or PDA, but may alternatively comprise any other device that is capable of electronic communications and programming.

The external user interface 270 may comprise a programming device 270a that is capable of programming various modules and stimulation parameters of the IMD 200. In one embodiment, the programming device 270a is capable of executing a data-acquisition program. The programming device 270a may be controlled by a medical professional, such as a physician, at a base station in, for example, a doctor's office. The programming device 270a may download various parameters and program software into the IMD 200 for programming and controlling its operation. The programming device 270a may also receive and upload various status conditions and other data from the IMD 200.

The communication user interface 260 may comprise hardware, software, firmware, and/or any combination thereof. Communications between the external user interface 270 and the communication user interface 260 may occur via a non-invasive, wireless or other type of communication, illustrated generally by line 275 in FIG. 2. Various software and/or firmware applications may be loaded into the programming device 270a for programming the external user interface 270 for communications with the IMD 200. In one embodiment, the external user interface 270 may be controlled by Windows® CE operating system offered by Microsoft Corporation of Redmond, Wash.

In one aspect of the present invention, a neurostimulation system generates a first electrical signal having a plurality of parameters including a primary time period and an off-time, and applies the first electrical signal to a nerve. During the off-time of the first electrical signal, the system generates a second electrical signal and delivers the second signal to the nerve. In some embodiments, both the first and second electrical signals are pulsed electrical signals further defined by a current magnitude, a pulse width, and a frequency. Preferably, at least one of the current magnitude, the pulse width, and the frequency of the second electrical signal is less than that of the first electrical signal. In some embodiments the frequency of the second electrical signal is less than 10 percent of the frequency of the first electrical signal. In some embodiments, the current of the second electrical signal is less than 75% of the magnitude of the current of the first electrical signal. In some embodiments, the pulse width of the second electrical signal is less than 75% of the magnitude of the pulse width of the second electrical signal.

In another aspect of the present invention, a neurostimulation system generates a first electrical signal defined by a primary time period and an off-time. The system also provide a second electrical signal having a secondary time period (which comprises an on-time and optional ramp-up and ramp-down signals for the second signal), and a secondary off-time. At least a portion of the secondary time period of the second electrical signal occurs during the off-time of the first electrical signal. In a more particular embodiment, the second electrical signal comprises an on-time that occurs entirely during the off-time of the first electrical signal. In a still more particular embodiment, the on-time of the second electrical signal is the same as the off-time of the first electrical signal. In an even more particular embodiment, the off-time of the second electrical signal is the same as the primary time period of the first electrical signal.

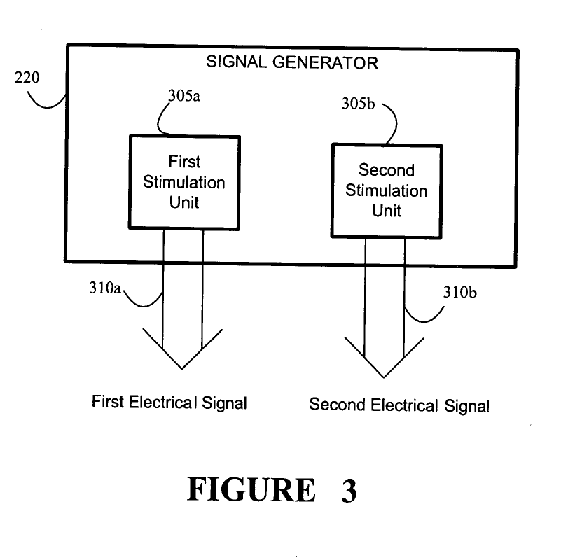

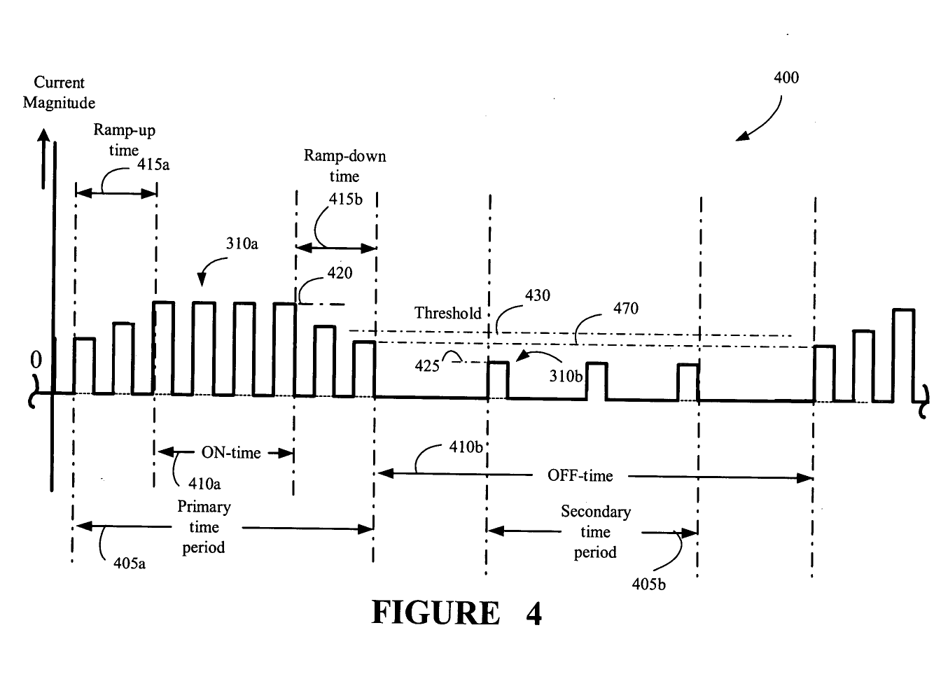

Referring to FIG. 3, a particular embodiment of the electrical signal generator 220 of FIG. 2 is shown, with a first stimulation unit 305a to generate a first electrical signal 310a, and a second stimulation unit 305b to generate a second electrical signal 310b. In another embodiment (not shown), electrical signal generator 220 may be capable of generating both the first and second electrical signals 310a, 310b from a single stimulation unit. Various types of stimulus signals may be generated by the first and second stimulation units 305a, 305b with different signal characteristics based on separate sets of parameters that define the first and second electrical signals 310a, 310b, respectively. Preferably, both parameter sets may be programmed into IMD 200 by external user interface 270. An example of a composite stimulation signal for treatment of a medical disorder, including first and second electrical signals 310a, 310b, is illustrated in FIG. 4 and described hereinafter.

FIG. 4 depicts a stylized representation of a composite electrical stimulation signal 400, which comprises first and second electrical signals 310a, 310b, in accordance with one illustrative embodiment of the present invention. The IMD 200 shown in FIG. 2 may use the electrical signal generator 220 to generate the composite stimulation signal 400 to stimulate a nerve of a patient. For example, the implantable medical device 100 may generate and apply to the nerve the first electrical signal 310a during a primary time period 405a comprising a ramp-up time 415a, a treatment on-time 410a and a ramp-down time 415b. The second electrical signal 310b is applied to the nerve during a secondary time period 405b corresponding to at least a portion of the off-time 410b of the first signal 310a. In one embodiment, the nerve or the portion of the nerve may comprise a selected cranial nerve such as the vagus nerve. By applying a stimulation signal 400 comprising both first and second electrical signals 310a, 310b, the implantable medical device 100 may provide a desired therapy to the patient for treating a disorder or a medical condition.

To condition a nerve or the brain of the patient, the primary time period 405a of the first electrical signal 310a may comprise one or more sub-periods such as a ramp-up period 415a, during which a pulsed signal that increases in current magnitude is provided; the on-time period 410a comprising a pulsed, constant current; and a ramp-down period 415b, in which the current decreases in magnitude. As shown in FIG. 4, the second electrical signal 310b may comprise a constant current signal having a reduced current magnitude and frequency relative to the first electrical signal 301a. Although both the first electrical signal 310a and the second electrical signal 310b are shown in FIG. 4 as being defined by a plurality of non-random parameters, one or more parameters of either or both of the first and second electrical signals may be randomized, as described more fully in co-pending U.S. patent application Ser. No. 11/193,520 (Enhancing Intrinsic Neural Activity Using a Medical Device to Treat a Patient), and Ser. No. 11/193,842 (Medical Devices For Enhancing Intrinsic Neural Activity), each filed in the name of Randolph K. Armstrong and assigned to the assignee of the present application. The entirety of each of the '520 and '842 applications is hereby incorporated herein by reference.

Referring again to FIG. 4, the IMD 200 may apply the second electrical signal 310b for a secondary time period that is a predefined portion of the off-time 410b of the first electrical signal 310a. In one embodiment, the predefined portion may end before the primary time period 405a begins, i.e., the second electrical signal 310b may be applied for only a portion of the off-time 410b of the first electrical signal 310a. In another embodiment, the predefined portion may be substantially the entire off-time 410b of first electrical signal 310a. In alternative embodiments not shown in FIG. 4, the secondary time period 405b may partially overlap primary time period 405a, and the first electrical signal 310a may overlap the second electrical signal 310b, as set forth below.

The electrical signal generator 220 may provide a second electrical signal 310b having a low frequency relative to the first electrical signal 310a. Alternatively, the second electrical signal 310b may comprise a low current magnitude relative to the first electrical signal 310a. Without being bound by theory, providing a second electrical signal 310b during an off-time 410b for the first electrical signal 310a may reduce discomfort experienced during the first signal by conditioning the nerve prior to the start of the first signal 310a.

In some embodiments, one or more parameters defining the second signal (e.g., current magnitude, frequency, pulse width, and the length of the secondary time period itself) may be determined as part of a feedback system in which the IMD 200 detects a body parameter of interest. The body parameter sensor may provide an indication to substantially turn off a primary therapeutic stimulation function of the IMD 200, and the IMD 200 may in response set one or more parameters defining the second electrical signal as a fraction or multiple of the corresponding parameter of the first electrical signal 310a, such that second electrical signal 310b is provided at a level 425 that is below a predetermined threshold 430. The given threshold 430 may be a sub side-effect level. Thus, the IMD 200 may provide an option to bring the output level 420 down to the sub-side-effect level instead of completely turning off the therapeutic stimulation function of the IMD 200 during the secondary time period 405b. In other embodiments, the parameters defining the second electrical signal 310b may not be determined by a feedback signal.

By separately generating the second electrical signal 310b and the first electrical signal 310a, in one embodiment, the IMD 200 may provide for overlaying the second electrical signal 310b during at least a part of the primary time period 405a. The IMD 200 may programmably adjust one or more parameters of the first electrical signal 310a and/or the second electrical signal 310b during their respective time periods based on a sensed body parameter.

Referring simultaneously to FIGS. 3 and 4, the second unit 305b may provide the second electrical signal 310b to apply a low-level signal relative to the first electrical signal 310a during the secondary time period 405b for at least a portion of the off-time 410b of first signal 310a. To provide the second electrical signal 310b during the secondary time period 405b, the second stimulation unit 305b may use the therapeutic data 212 that includes programmable parameter data. In an alternative embodiment (not shown) the secondary time period 405b may exceed the off-time 410b of the first electrical signal. In another embodiment, the secondary time period 405b may be the same as the off-time for the first electrical signal 310a. In a still further embodiment, the secondary time period 405b may be substantially smaller than the off-time 410b or the on-time 410a.

The IMD 200 may programmably change the primary time period 405a (including ramp-up, on-time and/or ramp-down time) and the off-time 410b of the first electrical signal 310a, as well as the secondary time period and off-time of the second electrical signal 310b, to provide a wide variety of composite electrical signals 400. The IMD 200 may modulate one or more parameters (e.g., a current magnitude, a pulse period, a polarity, and a pulse width, etc.) of first and/or second electrical signals 310a, 310b by selectively varying at least one parameter of the parameters associated with the first or the second electrical signals.

The IMD 200 may stimulate the nerve with the second stimulus signal 310b at a frequency that modulates a nerve receptor. For example, the nerve, such as a cranial nerve of the patient, may be stimulated to maintain a therapeutic effect of the first electrical signal 310a during the secondary time period 405b. The IMD 200 may stimulate the nerve at a sub-threshold level that causes the second electrical signal 310b to remain below a perception level 470 of the patient during the secondary time period 405b. At the same time, the second electrical signal 310b may provide benefits, such as an increase in the efficacy of a stimulation therapy, a reduction in side effects, and/or an increase in tolerability to higher levels of stimulation.

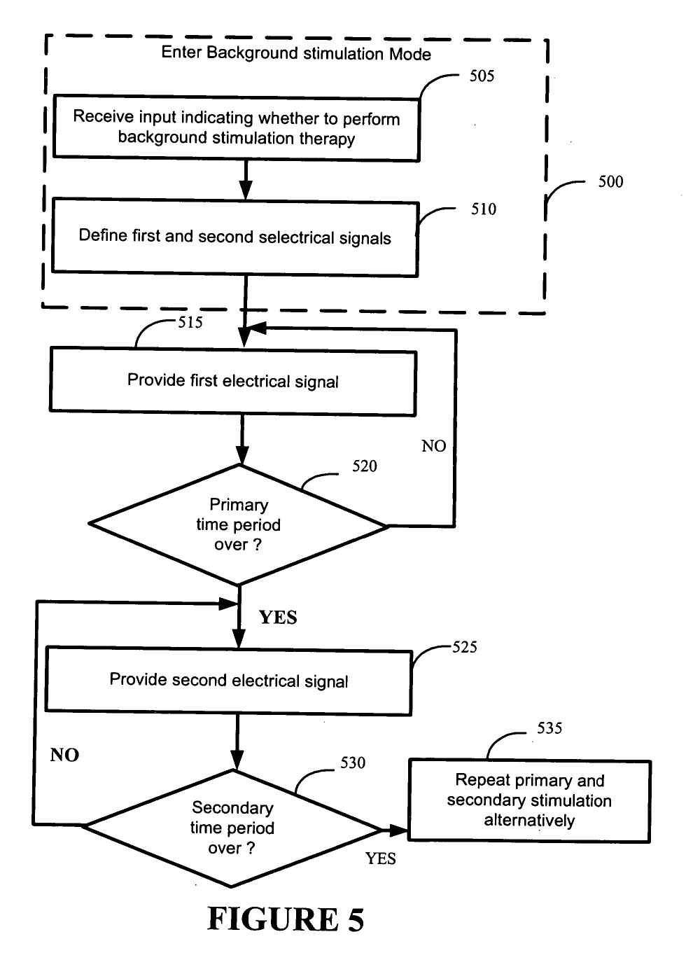

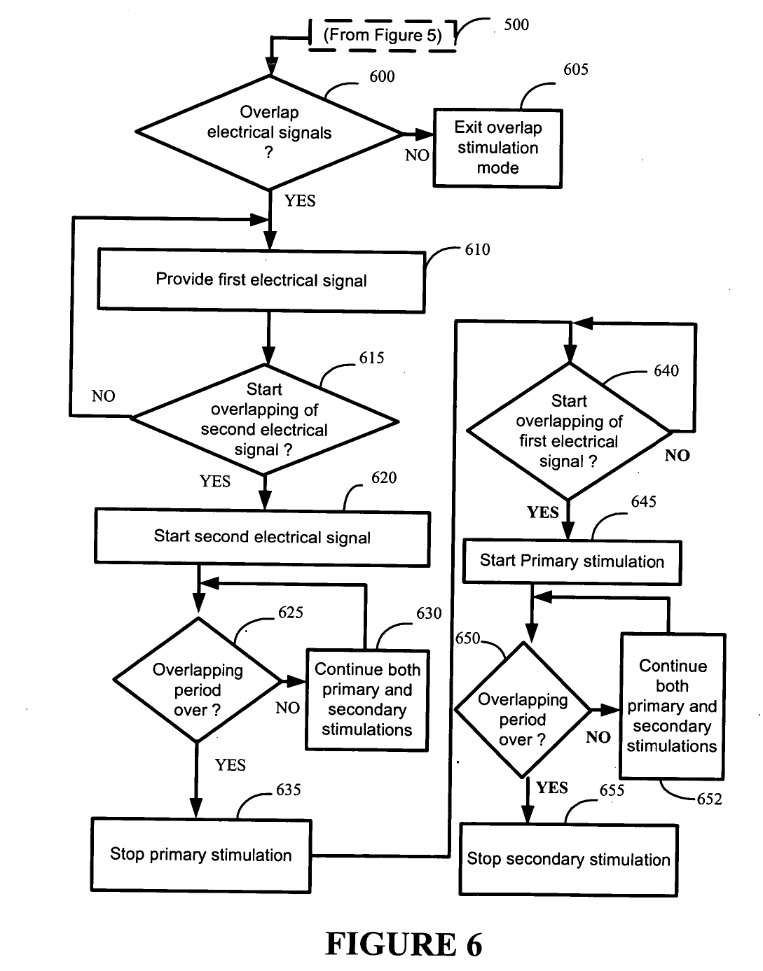

Turning now to FIG. 5, a flowchart depiction of a method of providing first and second electrical signals from the IMD 200, in accordance with one illustrative embodiment of the present invention, is provided. At block 500, the IMD 200 may enter a background stimulation mode in which a background electrical signal is provided in addition to a primary electrical signal. Initially, the IMD 200 may receive the therapeutic data 212 input, indicating whether to perform a background stimulation therapy that affects a disease state of the patient, as shown in block 505. For example, the IMD 200 may receive the therapeutic data 212 to provide a stimulation therapy that affects a disease state of the patient, wherein the therapy includes stimulation during a primary period and a secondary period. Using the therapeutic data 212, the IMD 200 may define the first and second electrical signals 310a, 310b, as shown in block 510. Defining the first and second electrical signals 310a, 310b may include defining the primary and secondary time periods 405a, 405b, in addition to other parameters (e.g., a current magnitude, a pulse period, a polarity, and a pulse width, etc.) relating to the signals.

To apply a therapeutic stimulus signal to a nerve of a patient during a first time period, i.e., the primary time period 405a, at block 515, the IMD 200 may provide the first electrical signal 310a. A check at a decision block 520 determines whether the primary time period 405a for stimulation or treatment has lapsed. If the primary time period 405a has lapsed, the IMD 200 provides the second electrical signal 310b, at block 525. If the primary period has not lapsed, the IMD 200 continues to provide the first electrical signal 310a until the primary time period 405a ends. That is, the IMD 200 may enter in a non-therapeutic or a secondary therapeutic mode during a second time period subsequent to a first time period. In the secondary therapeutic mode, the IMD 200 may apply a background stimulus signal comprising the second electrical signal during at least a portion of the second time period.

A check at a decision block 530 determines whether the secondary time period 405b has lapsed. If the secondary time period 405b has lapsed, the IMD 200 may repeat the first and second electrical signals, at block 535. If the secondary time period 405b has not ended, the IMD 200 continues to provide the second electrical signal 310b until the secondary time period 405b ends.

Based on the therapeutic data 212, in another embodiment, the IMD 200 may alternatively stimulate a patient's nerve with the first electrical signal 310a during the primary time period 405a and with the second electrical signal 310b during the secondary time period 405b for a given overall treatment period of a stimulation therapy. In a particular embodiment, the IMD 200 may provide electrical neurostimulation therapy to the patient such that the second electrical signal 310b comprises a pulsed electrical signal defined by a plurality of parameters, such as a current magnitude, a pulse period, a polarity, and/or pulse width, with at least one of the parameters comprising a random value. In this embodiment, the IMD 200 may randomly vary the current magnitude, pulse period, polarity, and/or the pulse width of adjacent pulses during the secondary time period within defined limits. In a more specific embodiment, both the current magnitude and the pulse width of electrical pulses in the second electrical signal 310b may vary randomly during the secondary time period 405b. As one example, the current magnitude for each pulse may randomly vary from 0.25 to 1.50 milliamps, and the pulse width for each pulse may randomly vary from 50 microseconds to 750 microseconds.

The electrical signal generator 220 may generate the first and the second electrical signals 310a, 310b for delivery to a selected portion of a selected nerve of a patient. The controller 215 operatively coupled to the electrical signal generator 220 may be adapted to apply the first electrical signal 310a to the selected nerve of the patient during the primary time period 405a. The controller 215 may apply the second electrical stimulus signal 310b to the selected nerve of the patient during the secondary time period 405b in which the first electrical stimulus signal 310a is off.

In this manner, the IMD 200 may stimulate the selected portion of the selected nerve of the patient with a predetermined sequence of electrical pulses from the electrical signal generator 220 applied to the selected nerve. To affect a disease state, the IMD 200 may provide a reduced therapeutic stimulation relative to the first electrical signal 310a during the secondary time period 405b. The IMD 200 may stimulate the nerve of the patient with the second electrical signal 310b based on the therapeutic data 212 at a frequency that aids in maintaining a therapeutic effect and/or eliminating or reducing side effects associated with the first electrical signal 310a during the secondary time period 405b.

The first and second electrical signals 310a, 310b may be defined based on a plurality of parameters, e.g., a current magnitude, a pulse period, a polarity, and/or a pulse width. The second electrical signal 31b may stimulate a portion of a nerve at a sub-threshold level that is below the perception level 470 of the patient during the secondary time period 405b.

Referring to FIG. 6, a flowchart depiction of an embodiment of a method for providing overlaid stimulation using the implantable medical device of FIG. 2 is provided. In this embodiment, the IMD 200 may employ the first and second electrical signals 310a, 310b in an overlapping fashion. A background stimulation mode may be initiated (block 500). A check at a decision block 600 determines whether an overlap of the primary and secondary time periods 405a, 405b is indicated for a stimulation or a treatment therapy. If the therapeutic data 212 indicates an overlaid stimulation mode with the primary and secondary time periods 405a, 405b at least partially overlaid, the first stimulation unit 305a may proceed to provide the first electrical signal 310a, as shown at block 610. If overlaid stimulation is not indicated, the IMD 200 may exit from the overlaid stimulation mode, as depicted in block 605. In one embodiment, upon exiting the overlaid stimulation mode, the IMD 200 may implement the background stimulation mode described in FIG. 5.

Referring again to FIG. 6, a check at a decision block 615 may determine whether to start the overlapping of the second electrical signal 310b with the first electrical signal 310a. Upon reaching an overlaid stimulation start point at which the primary and secondary time periods 405a, 405b overlap, the second stimulation unit 305b begins applying the second electrical signal 310b, as shown at block 620. If the time to begin overlapping the electrical signals/time periods has not arrived, the IMD 200 may continue to provide only the first electrical signal 310a, as depicted in block 610.

A check at decision block 625 may ascertain whether an end of an overlapping period of the first and second electrical signals 310a, 310b has been reached. Upon reaching the end of an overlapping period, the first electrical signal 310a is stopped, as shown in block 635. Conversely, if the overlapping period is not over, the IMD 200 continues to overlay the second electrical signal 310b over the first electrical signal 310a, as shown at block 630. This process may continue until the overlapping period has lapsed.

Subsequent to determining that the overlapping period has ended and the first electrical signal 310a has been stopped, at a decision block 640, the IMD 200 determines whether to start overlapping the first electrical signal 310a with the second electrical signal 310b. Upon reaching an overlap stimulation start point at which the primary and secondary time periods 405b, 405b overlap, the first stimulation unit 305a begins apply the first electrical signal 310a, as shown at block 645. Otherwise, at the decision block 640, the IMD 200 may continue to provide only the second electrical signal 310b, as depicted in block 620.

A check at a decision block 650 ascertains whether an end of an overlapping period of the first and second electrical signals 310a, 310b has been reached. Upon reaching the end of an overlapping period, the second electrical signal 310b is stopped, as shown in block 655. If the end of the overlapping period has not been reached, the IMD 200 continues to overlay the first electrical signal 310a over the second electrical signal 310b, as shown at block 652.

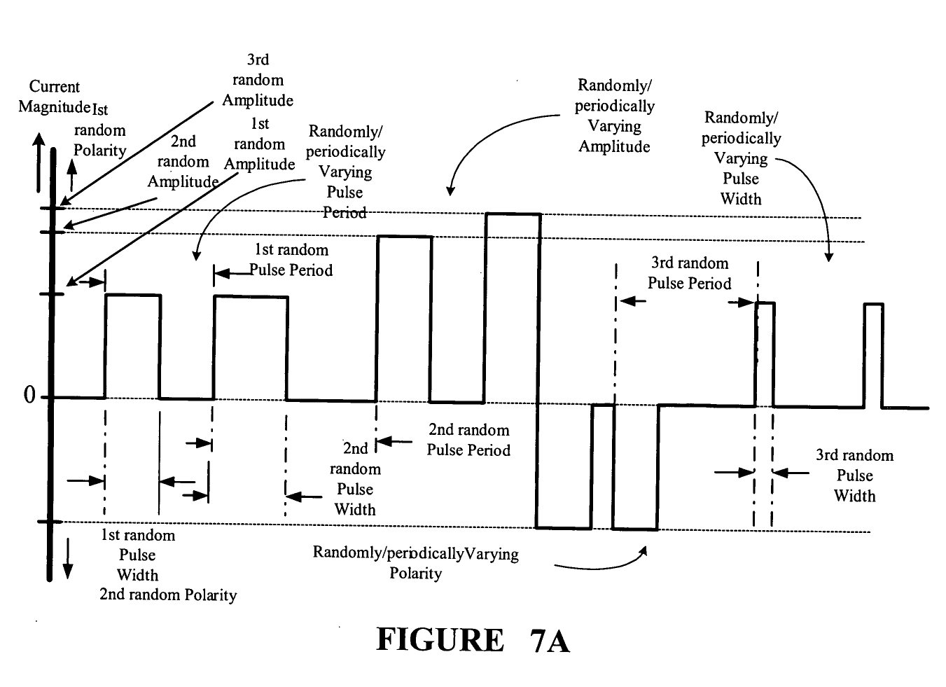

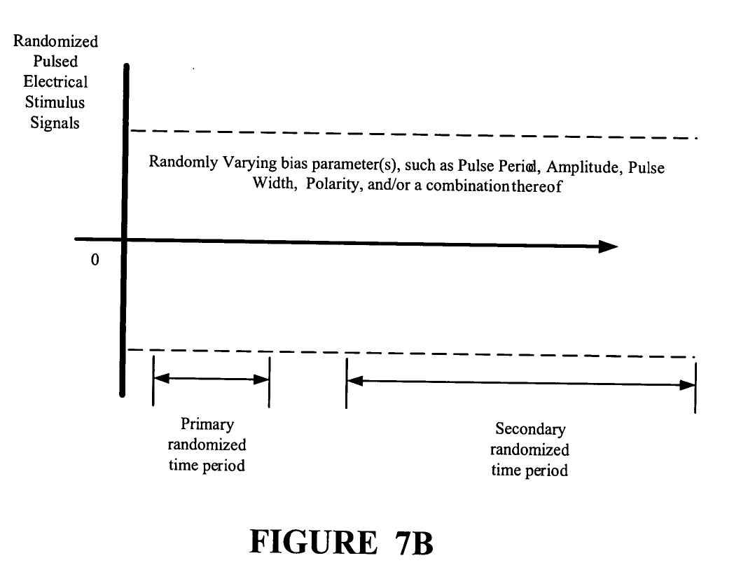

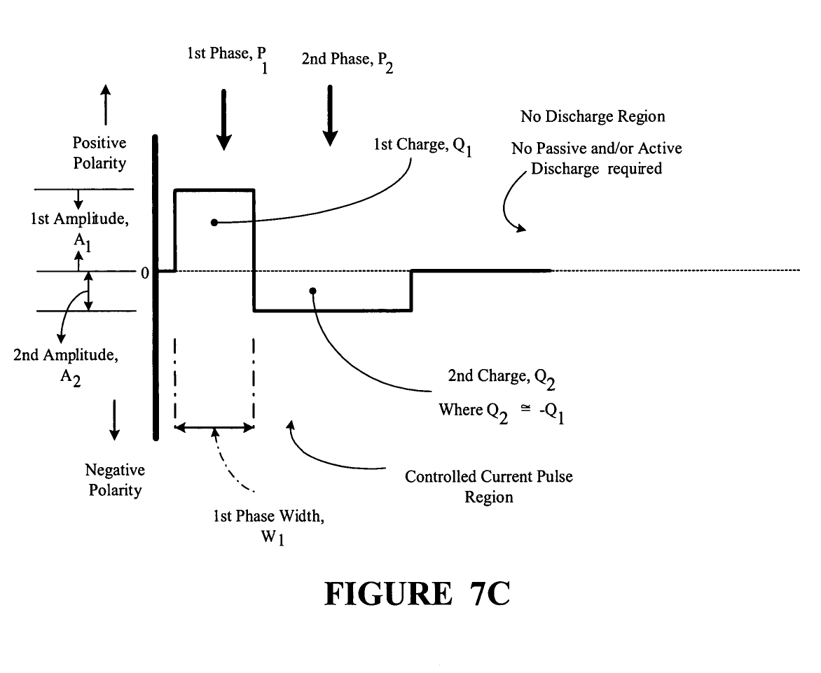

Referring to FIGS. 7A-7C, one embodiment of waveforms illustrates a pulsed first electrical signal 310a suitable for use in the present invention. The illustrations are presented principally for the sake of clarifying terminology for a plurality of parameters that may be used to define a pulsed electrical signal including a current amplitude, a pulse width, a pulse period (i.e., time interval between the start of adjacent pulses), and a pulse polarity, that may be used by the electrical signal generator 220 to generate a pulsed electrical signal. Other parameters (not shown) include signal on-time and signal off-time for non-continuous signals. In embodiments of the present invention, at least one of the voltage amplitude, current amplitude, pulse width, pulse period, pulse polarity, and (for non-continuous signals), signal on-time and signal off-time comprises a random value within a defined range. Examples of the defined range(s) for generating a desired stimulation based treatment therapy from the electrical signal generator 220 is described with reference to FIGS. 7A-7C, which illustrate the general nature, in idealized representation, of pulsed output signal waveforms delivered by the output section of the IMD 200 to electrode assembly 125. One or more biasing parameters may be randomly generated by the electrical signal generator 220 to generate a pulsed electrical signal that varies within a defined range.

A continuous signal, as used herein, refers to an electrical signal without a distinct on-time and off-time. A continuous signal may be delivered without a distinct on-time and off-time as either a pulsed signal having a constant or random pulse period or frequency, or as a purely continuous signal with no break in current flow (although other parameters, such as current magnitude and polarity, may vary within the signal). A non-pulsed signal, as used herein, refers to a signal in which a current is always being delivered during the on-time period, as distinct from a pulsed signal in which flow of current during an on-time period is separated by short periods (typically milliseconds or seconds) of no current flow. It should be noted that non-pulsed signals may be delivered according to a programmed or random on-time and off-time (for example, to allow a recovery/refractory period for the neural tissue stimulated). However, unless the on-time periods have breaks in current flow within each on-time period, the signal remains a non-pulsed signal as used herein.

FIG. 7A illustrates an exemplary pulsed electrical stimulus signal provided by embodiments of the present invention. The electrical stimulus signal may be a non-continuous signal defined by an on-time and an on-time, or may comprise a continuous signal (i.e., a signal that does not comprise a distinct on-time and off-time) without discrete pulse bursts. The electrical stimulus signal may alternatively comprise a non-pulsed signal (which may be continuous or non-continuous) with no current breaks during a stimulation period. Whether continuous or non-continuous, in one embodiment the invention comprises signals in which one or more stimulus signal parameters are randomly changed for particular pulses in a pulse train (pulse-to-pulse randomization), or alternatively for pulses in adjacent pulse trains (burst-to-burst randomization). Burst-to-burst randomization may comprise changing only the on-time and/or off-time, in which case each of the pulses may be non-random as defined by any of voltage, current, pulse width, pulse period, or frequency, but the duration of adjacent pulse bursts or the interval separating them may comprise a random time interval.

In particular, as FIG. 7A illustrates, the electrical signal pulses in the a pulsed electrical stimulus current signal provided by the IMD 200 may randomly vary in current amplitude, as shown by pulses having first, second and third random amplitudes, respectively, and/or in pulse widths as illustrated by the pulses having first, second and third random pulse widths, respectively. For example, current magnitude of the pulses may be random and vary within any arbitrarily defined range within the range of from −8.0 milliamps (mA) to 8.0 milliamps, such as from −3.0 to 3.0 milliamps or from 0.25 to 1.5 milliamps, with optional charge-balancing. Similarly, pulse widths may be random and vary within any arbitrarily defined range within the range of 1 microsecond to 1 second, such as from 50 to 750 microseconds, or from 200 to 500 microseconds.

In addition to current magnitude and pulse width, FIG. 7A further shows that in some embodiments pulse polarity may vary randomly between a first polarity, indicated by the pulses having a peak above the horizontal zero current line, and a second, opposite polarity, indicated by a peak below the zero current line. FIG. 7A omits, for convenience, any charge-balancing component for a particular pulse. However, it will be understood that each pulse may include a passive or active charge-balancing component. FIG. 7A further illustrates that pulse periods of the electrical pulses also may vary randomly, as illustrated by adjacent pulse pairs having first, second and third random pulse periods. For example, pulse periods of the pulses may be random and vary randomly within any arbitrarily defined range within the range of 1 microsecond to 1 second, for example from 50 microseconds to 200 milliseconds.

While not shown in FIG. 7A, for non-continuous electrical stimulus signals defined by an off-time and an on-time, one or both of the on-time and off-time may vary randomly within defined ranges. For example, the on-time defining a pulse burst (or a non-pulsed signal) may be random and vary randomly within any arbitrarily defined range within the range of 1 second to 24 hours and the off-time defining a pulse burst or non-pulsed signal may also be random and vary randomly within any arbitrarily defined range within the range of 1 second to 24 hours.

While FIG. 7A describes parameter randomization for a pulsed electrical stimulus signal, similar randomization of parameters may be provided for a non-pulsed electrical biasing signal. In particular, while not defined by a pulse width or a pulse interval, a non-pulsed signal may nevertheless be defined by one or more of a current amplitude and a current polarity, and a non-continuous non-pulsed signal may further be defined by an ON-time and an OFF-time. One or more of the foregoing parameters may be randomized for a non-pulsed signal, in similar manner to that described for a pulsed signal, supra.

FIG. 7B and illustrates that first and second electrical signals 310a, 310b may comprise a randomized signal for a first period of time and non-randomized signals for a second period of time. A stimulus parameter for one or both of the first and second electrical signals 310a, 310b may comprise a random value on a pulse-to-pulse basis and vary within a defined range across a random and/or periodic time interval, but otherwise is non-random. For example, pulse period, amplitude, pulse width, polarity, and/or a combination thereof may randomly vary within a defined range for a first time interval ranging from 1 second to 24 hours. One or more stimulus parameters may be randomly varied in first and second periodic ranges during the first time period. For example, the pulse period may be varied randomly for a 30 second period at a value from 50 microseconds to 750 microseconds. In a second time period, the pulse period may comprise a non-random value, for example 500 microseconds for a period of 1 minute. In other embodiments, the ranges of the randomization parameters may comprise a split range. For example, the current magnitude may be allowed to vary on a pulse-to-pulse basis within the ranges of 0.25 to 0.75 milliamps and also in the range of 1.25 to 1.50 milliamps. Accordingly, the current may comprise any value between 0.25 milliamps and 1.50 milliamps except for values comprising 0.76 milliamps to 1.24 milliamps. Such split range randomization may be beneficial for some patients, and is considered to be within the scope of the present invention.

The randomized electrical stimulus current signal provided by the IMD 200 may be directed to performing selective activation of various electrodes (described below) to target particular tissue for excitation. An exemplary randomized electrical stimulus current pulse signal provided by the IMD 200 is illustrated in FIG. 7A, where randomly varying polarity of a pulse signal is illustrated. In one embodiment, the randomly varying polarity may be employed in conjunction with alternating electrodes for targeting specific tissues.

FIG. 7C illustrates an exemplary randomized electrical signal pulse that provides various random phases that correspond to a change in amplitude and a change in polarity. As described above, a phase of a pulse may randomly take on various shapes and current levels, including a current level of zero amps. In one embodiment, a phase with zero current may be used as a time delay between two current delivery phases of a pulse.

More specifically, FIG. 7C illustrates a randomized electrical signal pulse and having a first phase with a first random amplitude relating to a first charge, Q₁, and a second phase that corresponds to a second random amplitude relating to a second charge, Q₂. In the signal illustrated in FIG. 7C, the second charge Q₂ is substantially equal to the negative value of the first charge Q₁. Therefore, the charges, Q₁ and Q₂, balance each other, reducing the need for active and/or passive balancing of the charges. Hence, the signal pulse illustrated in FIG. 7C is a charge-balanced, randomized electrical signal pulse. Reducing the need for performing active and/or passive charge balancing may provide various advantages, such as power savings from the reduction of charge discharge, fewer circuit requirements, and the like. For example, applying the first and/or second electrical signals 310a, 310b may comprise applying a series of charge-balanced pulses (i.e., pulse bursts) for balancing an electrical charge resulting from the electrical signals. The current magnitude of the pulses may be random and vary within any arbitrarily defined range within the range of −8.0 milliamps to 8.0 milliamps, or may be non-random and programmably defined.

Various other pulse shapes may be employed in the randomized electrical biasing signal concepts provided by embodiments of the present invention and remain within the scope and spirit of the present invention. Use of the IMD 200 may improve efficacy of the vagus nerve stimulation (VNS) therapy in many neurological or neuropsychiatric conditions. In one embodiment, the second electrical signal 310b may comprise providing, during the off-time 410b of the first electrical signal 310a, a pulse burst in which the pulses have the same constant current magnitude and constant pulse width as the first electrical signal 310a, but at a frequency of 5 Hz or less. In another embodiment, the current magnitude of the second electrical signal pulses is below a perception threshold of the patient. By providing such a second electrical signal 310b, the current magnitude of the first electrical signal 310a may be able to be reduced without loss of efficacy and with reduced discomfort. Many prior art neurostimulators allow a patient to manually turn off the electrical signal (which may be done using, e.g., a magnet), typically to avoid an undesired side effect. In embodiments of the present invention, instead of causing the IMD 200 to completely turn off the electrical signal, a programmable or user option may provide a second electrical signal 310b having a reduced level of stimulation, e.g., below a perception threshold.

In one embodiment, providing a second electrical signal 310b at a reduced level during the off-time 410b of the first electrical signal 310a allows the duration of the off-time 410b to be increased without significantly decreasing efficacy of a therapy or a treatment. Increasing the duration of the off-time 410b period may provide reduced energy consumption of the battery in IMD 200. Providing a second electrical signal 310b may, in another embodiment, increase the patient's tolerance for higher current magnitudes for the first electrical signal 310a. In another embodiment, the second electrical signal 310b may reduce or eliminate a need for ramp-up and ramp-down periods. As a result, the IMD 200 according to embodiments of the present invention may improve efficacy of the therapy, increase longevity of a medical device, and/or reduce side effects of stimulation in the patient's body.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed is:

1. A method of providing a neurostimulation signal to a target body area of a patient using a medical device, comprising:
applying a first electrical signal defined by a first plurality of parameters to said target body area of the patient during a primary time period; and
applying a second electrical signal defined by a second plurality of parameters to said target body area of the patient during at least a portion of a secondary time period in which said first electrical signal is not applied.

2. The method of claim 1, wherein said first and second plurality of parameters each comprise at least one of a current magnitude, a pulse width, a frequency, a pulse period, an on-time and an off-time, and wherein applying a second electrical signal further comprises:
applying an electrical signal having a value of at least one parameter of said second plurality of parameters that is less than the same parameter of said first plurality of parameters.

3. The method of claim 1, wherein said first plurality of parameters comprises a current magnitude, a pulse width, a frequency, a pulse period, an on-time and an off-time and wherein said second electrical signal is applied during at least a portion of said off-time of said first electrical signal.

4. The method of claim 1, wherein applying a second electrical signal further comprises:
applying said second electrical signal for a predefined portion of said secondary time period.

5. The method of claim 4, wherein said predefined portion of said secondary time period ends before said primary time period begins.

6. The method of claim 5, wherein said predefined portion is substantially the entire said secondary time period.

7. The method of claim 1, wherein said first plurality of parameters comprises a first frequency and said second plurality of parameters comprises a second frequency less than said first frequency.

8. The method of claim 1, wherein said first plurality of parameters comprises a first current magnitude and said second plurality of parameters comprises a second current magnitude less than said first current magnitude.

9. The method of claim 1, further comprising:
detecting a signal to turn off said medical device; and
in response to said indication, turning off said first electrical signal and applying said second electrical signal.

10. The method of claim 1, wherein said second electrical signal is below a target threshold.

11. The method of claim 10, wherein said target threshold is a sub-side-effect threshold.

12. The method of claim 1, wherein applying said second electrical signal further comprises applying said signal during at least a portion of said secondary time period in which said first stimulus signal is applied.

13. The method of claim 1, further comprising:
programmably adjusting at least one parameter of said first plurality of parameters based on a sensed body parameter.

14. The method of claim 1, wherein at least one of said primary time period and said secondary time period comprises a random time period.

15. The method of claim 1, wherein said first and said second plurality of parameters each comprises an on-time and wherein said on-time of said first electrical signal is less than said on-time of said second electrical signal.

16. The method of claim 1, wherein said second electrical signal produces an effect selected from the group consisting of improving a therapeutic effect produced by said first electrical signal and reducing a side effect associated with said first electrical signal.

17. The method of claim 10, wherein said second electrical signal is below the perception level of the patient.

18. The method of claim 1, wherein said target body area is a cranial nerve of the patient.

19. The method of claim 1, further comprising:
alternating said steps of applying said first electrical signal and applying said second electrical signal.

20. The method of claim 1, wherein said second plurality of parameters comprises a current magnitude, a pulse width, an on-time and an off-time, and wherein at least one of said current magnitude, and said pulse width, said on-time and said off-time varies randomly within defined limits.

21. The method of claim 20 wherein both said current magnitude and said pulse width vary randomly within a pulse burst, said current magnitude for each pulse randomly varying within a range within the range of from 0.25 to 1.50 milliamps, and said pulse width for each pulse randomly varying within a range within the range of from 50 microseconds to 750 microseconds.

22. A neurostimulator for treating a patient with a medical condition comprising:
a stimulus generator to generate a first and a second electrical signal for delivery to a selected nerve of a patient; and
a controller operatively coupled to said stimulus generator, said controller being adapted to apply said first electrical stimulus signal to the selected nerve of the patient during a primary time period, and to apply said second electrical stimulus signal to the selected nerve of the patient during at least a portion of a secondary time period in which said first electrical stimulus signal is off.

23. A method of providing an electrical neurostimulation therapy using a medical device, comprising:
applying a first electrical signal to a nerve of a patient during a first time period; and
applying a second electrical signal to the nerve of the patient during a second time period in which said first electrical signal is off.

24. The method of claim 23, wherein said second electrical signal provides a reduced level of stimulation relative to said first electrical signal.

25. The method of claim 23, further comprising alternating said steps of applying said first electrical signal and applying said second electrical signal.

26. The method of claim 23, wherein said second electrical signal produces an effect selected from the group consisting of improving a therapeutic effect produced by said first electrical signal and reducing a side effect associated with said first electrical signal.

27. The method of claim 23, wherein said first and said second plurality of parameters each comprises an on-time, and wherein said on-time of said first electrical signal is less than said on-time of said second electrical signal.

28. The method of claim 29 wherein said second electrical signal modulates the electrical activity of the nerve at a level that is below the perception level of the patient.

29. In a method of providing an electrical signal to a cranial nerve of a patient characterized by providing an electrical signal defined by a plurality of parameters comprising at least a current magnitude, an on-time and an off-time, wherein said electrical signal is provided in repeating cycles in which the electrical signal is provided for an on-time period and not provided for an off-time period, the improvement comprising:
providing a second electrical signal, and applying said second electrical signal during at least one of said off-time periods.
